(19) United States
(12) Patent Application Publication
(10) Pub. No.: US 2026/0335546 A1
(43) Pub. Date: Oct. 29, 2026

(54) DYNAMIC ALLOCATION FOR MULTI-PUMP BOLUS VOLUMES

(71) Applicant: CareFusion 303, Inc., San Diego, CA (US)

(72) Inventors: Matthew Mark MICELI, San Diego, CA (US); Michael K. WORKMAN, Carlsbad, CA (US)

(21) Appl. No.: 19/190,436

(22) Filed: Apr. 25, 2025

(51) Int. Cl. A61M 5/172; G16H 10/60; G16H 20/17; G16H 80/00

(52) U.S. Cl. CPC A61M 5/172; G16H 10/60; G16H 20/17; G16H 80/00

(57) ABSTRACT

Certain aspects of the disclosure provide infusion systems and methods for generating an infusion plan for a set of infusion devices. For example, the method may include receiving an infusion order for a patient, determining the infusion order exceeds a bolus threshold, and identifying a set of infusion devices associated with the patient. The method may further include generating an infusion plan for the set of infusion devices based on the infusion order and adjusting one or more operational parameters of the set of infusion devices according to the infusion plan.

### BACKGROUND

### Field

Aspects of the present disclosure relate to allocation of infusion orders across a set of infusion devices.

### Introduction

An infusion pump is a type of medical device configured to infuse therapeutic fluid into a patient, such as in a hospital or other medical care site. Generally, an infusion pump is programmed to pump fluid at a specific flow rate and up to a specific volume in order to administer a specific dose of a treatment. In particular, the infusion pump may be configured to receive one or more operational parameters used to control operation of the infusion pump, often referred to as an infusion order. The one or more operational parameters may include one or more of flow rate, volume, velocity, infusion time, and/or the like. The infusion pump then operates according to the one or more operational parameters.

An infusion pump has mechanical operational limits based on the mechanisms for pumping, for example, a limit on the flow rate of the infusion pump due to the speed at which the pumping mechanism can pump fluid into the patient. The pumping mechanism may not be capable of pumping at a higher speed to achieve a high flow rate. Similarly, at low speeds, the pumping mechanism may become inaccurate, such that the infusion may be inconsistent or inaccurate because the pumping mechanism pumps too high of a volume of fluid with each pump.

Additionally, the infusion pump is coupled to a fluid container containing the fluid to be infused to the patient. Fluid containers may be a variety of shapes, sizes, and types. Example fluid containers may include syringes that contain the fluid for syringe pumps, bags for large-volume pumps, elastomeric reservoirs for elastomeric pumps, and the like. Different types and sizes of fluid containers may be used with different types of infusion pumps.

Thus, different infusion pumps may have different operation limits, such as flow rate, total infusion volume, and the like. In some cases, an infusion order may exceed an operational limit of the infusion pump, for example, a volume of the fluid in the fluid container coupled to the infusion pump or a flow rate of the infusion pump. In such cases, the infusion pump cannot be used to infuse the order.

### SUMMARY

Certain aspects provide a method comprising receiving an infusion order for a patient; determining the infusion order exceeds a bolus threshold; identifying a set of infusion devices associated with the patient; generating an infusion plan for the set of infusion devices based on the infusion order; and adjusting one or more operational parameters of the set of infusion devices according to the infusion plan.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1 depicts an example dynamic infusion allocation system.

FIG. 2 depicts an example workflow for dynamically allocating infusion orders across infusion devices.

FIG. 3 depicts an example workflow for generating infusion allocation plans.

FIG. 4 depicts an example modular infusion devices for allocation of an infusion order.

FIG. 5 depicts an example infusion system.

FIG. 6 depicts an example syringe infusion system.

FIG. 7 depicts an example method for dynamically allocating infusion orders across infusion devices.

FIG. 8 depicts an example computing device with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for allocation of infusion orders across a set of infusion devices.

An infusion order may prescribe a dose of the therapeutic fluid for administration to the patient, including indicating flow rate, volume, velocity, infusion time, and/or the like of the therapeutic fluid to infuse the dose to the patient. A clinician may set up an infusion device, such as an infusion pump, using the infusion order to infuse the dose to the patient. The clinician may select the infusion device based on the infusion order, for example, a large volume pump where the volume to be infused is large, or a syringe pump where a flow rate is low. In some cases, no single infusion device may be capable of infusing the infusion order, such as where the volume to be infused is greater than a single fluid container, or a flow rate is higher than the infusion pump's operational limits.

Further, some infusion orders may be a bolus dose. A bolus dose is a single dose of a therapeutic fluid given over a short period of time. For example, a bolus dose may be 200 mL of fluid A for 30 minutes. In either case, a regular infusion order or a bolus dose, the parameters of the infusion order may be beyond the operational capacity of the infusion pump, for example, beyond the operational threshold of the infusion pump.

Aspects of the present disclosure overcome such technical limitations by allocating an infusion order across one or more infusion devices, for example, where one infusion device is insufficient to effectuate the infusion order. Specifically, in certain aspects, a combination of two or more infusion devices may be used such that each infusion device infuses part of the infusion order, but all together, the combination of two or more infusion devices infuses the entire infusion order.

For example, an infusion order for 600 mL of a fluid to be infused for 30 minutes may be beyond the operational limits of a single infusion device, for example, beyond a flow rate threshold of 999 mL/hr because the flow rate of such an infusion order would be 1200 mL/hr. Aspects of the present disclosure enable dynamic allocation of the infusion order to two or more infusion devices, such that together the infusion order is accomplished. For example, a first infusion device may infuse 300 mL of the fluid for 30 minutes (e.g., a flow rate of 600 mL/hr, which is within the infusion device's operating capacity), while a second infusion device also infuses 300 mL of the fluid for 30 minutes. Thus, the combination of infusion devices infuses the patient with 600 mL of the fluid over 30 minutes.

In particular, certain aspects described herein determine whether a single infusion device is sufficient, such as where the infusion order may be accomplished by a single infusion device. If not, additional infusion devices coupled to the patient which could be used for the infusion order may be identified. Then, it is determined that a combination of infusion devices may be used to infuse the infusion order. The infusion order may then be allocated between the combination of infusion devices to generate an infusion plan, such that the operational parameters of each infusion device, when combined accomplish the infusion order. The operational parameters of each infusion device may be adjusted in accordance with the infusion plan to effectuate the infusion.

Furthermore, in some aspects, multiple infusion plans may be generated, for example, where different possible combinations of allocated operational parameters may achieve the infusion order. The multiple infusion plans may be ranked, such as based on a priority level, in accordance with priority rules, to maintain parity between infusion devices, or use up resources in an efficient manner, e.g., avoid waste.

In some aspects, modular infusion systems may be utilized in which multiple infusion devices may already be coupled to the patient, whereby a clinician does not need to add another device, saving clinician time and infusion supplies.

### Example Dynamic Infusion Allocation System

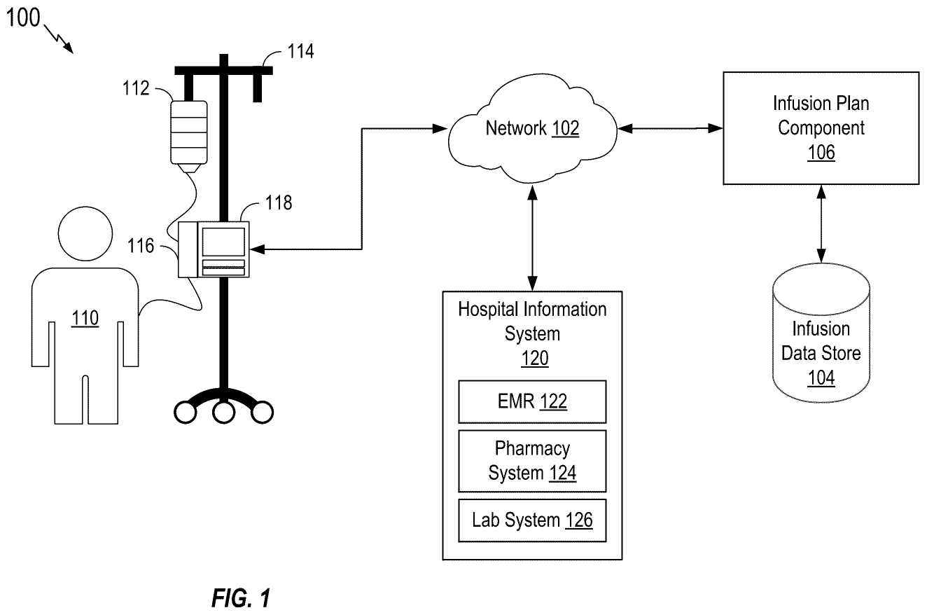

FIG. 1 depicts an example dynamic infusion allocation system 100 for generating infusion plans to allocate an infusion order across an infusion system, such as the infusion system 114, coupled to a patient 110.

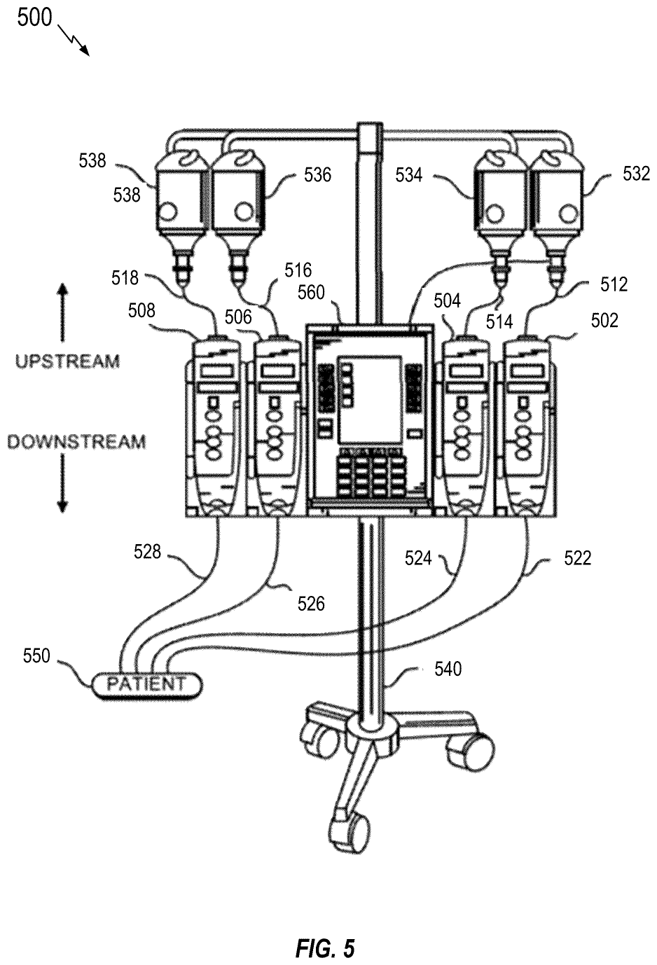

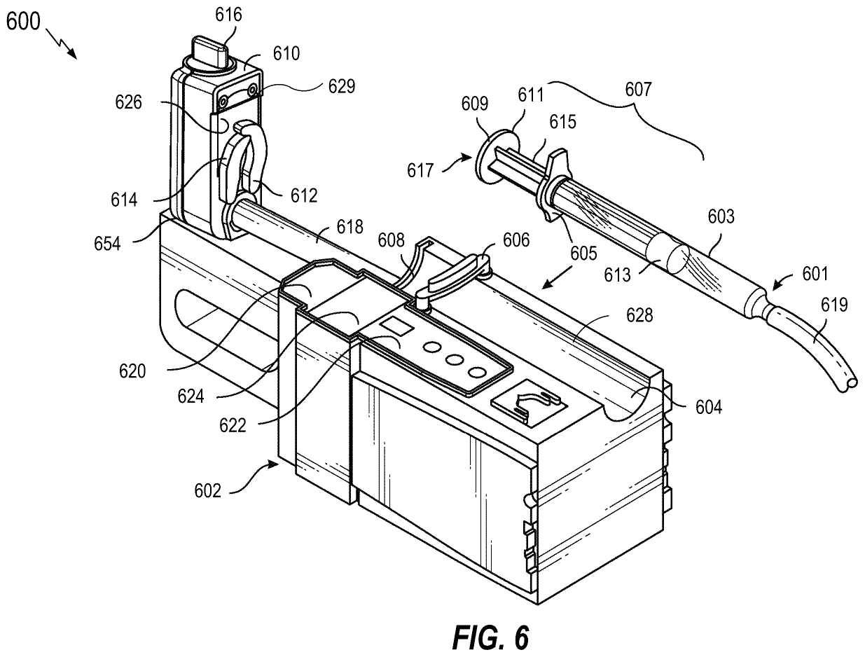

Infusion system 114 is an electromechanical device for infusing therapeutic fluids into the patient 110 in a controlled and precise manner. Exemplary infusion systems may include a large volume pump or a syringe pump, such as depicted in FIG. 5 or FIG. 6. Specifically, in this example, infusion system 114 includes one infusion pump 116. Infusion system 114 comprises an infusion pump controller 118 configured to control operations of infusion system 114. Infusion pump controller 118 may be an example of a pump control unit (PCU) or pump control device (PCD).

In general operation, therapeutic fluid is loaded into the infusion pump 116, for example, in a syringe, bottle, bag, or other fluid container 112, and the infusion system 114 is configured to pump, or infuse, the therapeutic fluid according to operational parameters of the infusion pump 116, including, for example, flow rate, volume, and/or time to administer a specific dose of treatment. As described in detail with respect to FIG. 5 or FIG. 6, below, the infusion pump controller 118 and/or the infusion pump 116 may include one or more buttons, controls, displays, keys, or the like to control operations of the infusion system 114. In some examples, a clinician may utilize the infusion pump controller 118 to enter operational parameters or otherwise control operation of the infusion pump 116. Further, the infusion pump controller 118 may display drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers.

In some aspects, the infusion pump controller 118 may contain a memory comprising a drug library or libraries, an event log or logs, and pump configuration settings, such as, but not limited to, profiles to be used in particular care areas such as ICU, PED, etc. The memory may be electronically loadable memory such as non-volatile memory (e.g., EEPROM). Drug libraries stored on infusion systems (which illustratively contain such information as the drug names, ranges of delivery parameter values such as proper concentrations, dosage units, and dose limits) may be used to perform drug calculation-based infusions in a clinical setting. A drug library stored within the infusion system may include clinical order settings such as limits set by the clinical institution for each drug of the library (also referred to as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward (e.g., a care area) of the medical institution in which the drug is being used (for example neonatal care unit (NICU), the intensive care unit (ICU), etc.), and depending on other factors. An alarm may be provided if the clinician sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

Infusion system 114 is further configured to transmit and receive data, such as data related to the infusion including infusion parameters, infusion data, operational data, or the like. Infusion system 114 may use network 102 to transmit and receive data. In some aspects, infusion system 114 is configured to transmit data to a hospital information system (HIS) 120 of a medical center of the infusion system 114.

For example, HIS 120 is configured with an electronic medical recordation (EMR) system 122, a pharmacy system 124, a laboratory system 126, or the like, for the medical center, to facilitate communications such as transmitting and receiving laboratory testing data, prescription data, documentation of a patient's medical history, diagnosis, treatments, laboratory data, medical device status, or the like. HIS 120 may include, or be configured to interface with, other hospital systems such as an admissions system, a billing system, a central supply system, unit station systems, or a medical decision support system.

In some aspects, the pharmacy system 124 includes a formulary and/or pharmacy information system. Pharmacy information systems may enable a safer physician medication order process. A pharmacy website (e.g., provided by the server) may provide the physician with a list of available drugs from which the physician may select. The pharmacy website may contain a drug library having the list of available drugs but may also contain and present to the physician the drug names associated with recommended dosages and dose limits that have been established or adopted by the medical center. In such a case where the physician need only select items from the computer screen rather than having to manually type in drug names and drug administration numbers (such as infusion rates, times, etc.) associated with administration of the medication, a more accurate medication process may result.

If a clinical order is for administration of a particular medication regimen, the order will be transmitted to the medical center's pharmacy information system 124. The pharmacy reviews the order, and once the order has been prepared, the order may be transmitted to a nurse station for matching with the appropriate patient. A formulary generally refers to an approved list of drugs for use (e.g., available to order for a patient) within a medical facility. Within a formulary, there may be indication for use information and/or concentrations and drug ranges approved for the medical center. A formulary may be used to define one or more medical device drug libraries, which may then be provided to infusion pumps within HIS 120. Inside the library, there is medication information such as drug names, concentration, diluent volume, strength, minimum or maximum infusion parameters for a drug, and other parameters. The establishment of these parameters, along with parameters for off-formulary orders, via the network 102, is useful for maintaining consistency across the medical center environment and ensuring an order is intelligible and executed according to expectations by other devices within the HIS 120 (e.g., an infusion system 114).

Where the clinical order is for a therapeutic fluid to be infused, e.g., with infusion system 114, the order may be called an infusion order, and prescribe a dose of the therapeutic fluid, and a time period for administration. In some aspects, an infusion order may further include operational parameters to infuse the dose in the time period prescribed.

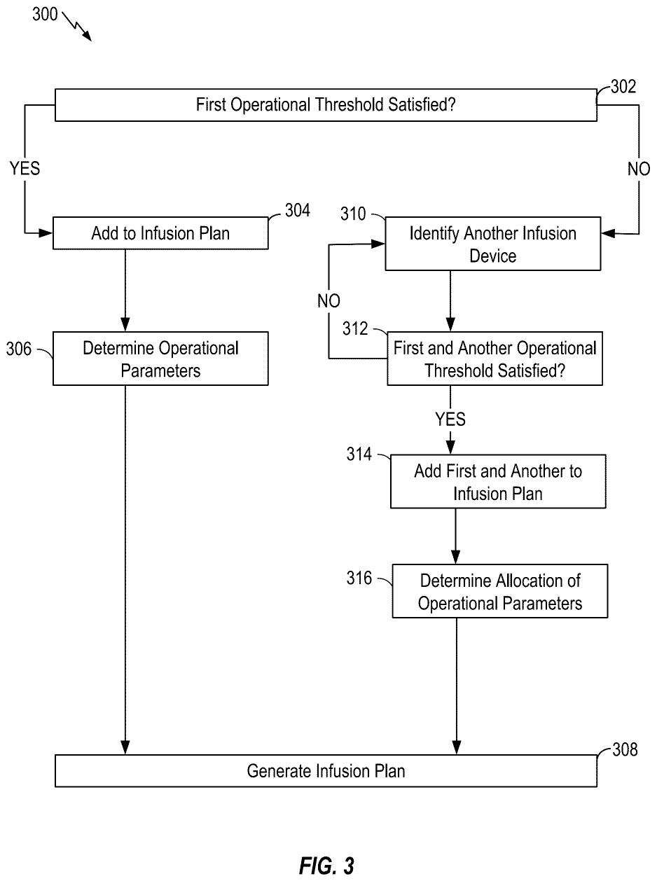

System 100 further includes an infusion plan component 106. Infusion plan component 106 is configured to generate an infusion plan to allocate an infusion order across a set of infusion devices, for example, one or more devices of infusion system 114 or one or more infusion systems coupled to the patient 110. Specifically, and as described further with respect to FIG. 3, an infusion plan may be generated to allocate an infusion order, such that two or more infusion devices operate in conjunction to effectuate infusion of the infusion order. An infusion plan may comprise an indication of one or more infusion devices for infusing the infusion order, as well as one or more operational parameters for infusing the infusion order. In some examples, a set of operational parameters for each infusion device of the infusion plan may be indicated in the infusion plan. The infusion plan may further comprise instructions for the indicated infusion device(s) to control operations of the infusion device(s) in accordance with the operational parameters. Infusion data, such as associated with the infusion plan, historical infusion data, infusion device data, or the like, is stored in infusion data store 104.

Although depicted here as separate devices, in some cases, the infusion plan component may be integral to or form part of infusion system 114 or HIS 120. In some cases, the infusion plan component 106 may form part of a clinician device, such as a mobile device, a nurse station monitoring device, a computing device, or the like.

The network 102 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

### Example Infusion Order Allocation Workflow

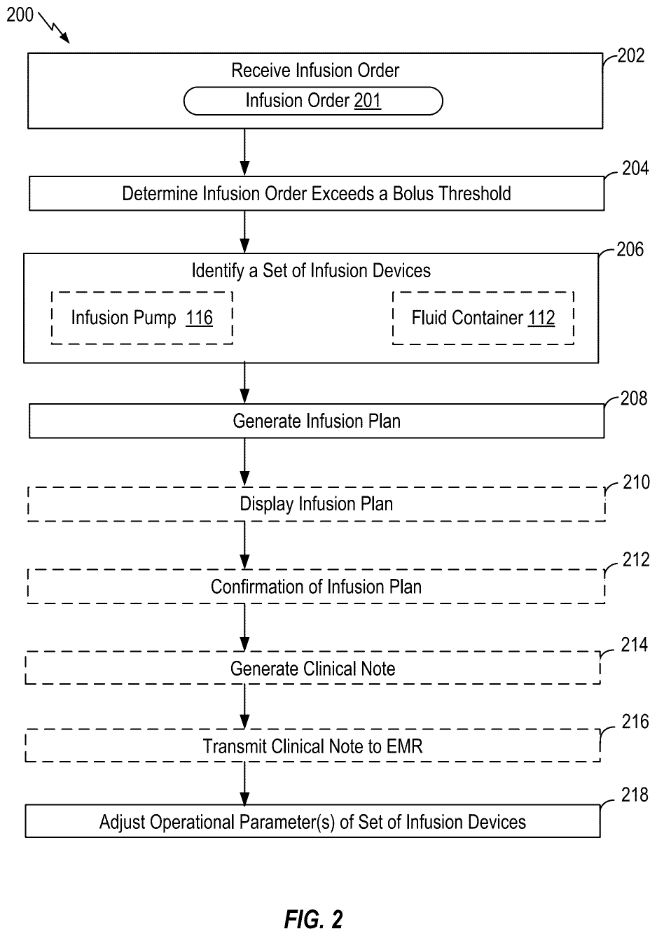

FIG. 2 depicts an example workflow 200 for dynamically allocating an infusion order across a set of infusion devices, for example, one or more devices of infusion system 114 or another infusion system coupled to the patient.

Initially, workflow 200 starts at block 202 with receiving an infusion order 201. In some cases, the infusion order 201 may be received from a clinician, for example, through pharmacy system 124 of HIS 120 in FIG. 1, inputted at an infusion system 114, such as through the infusion pump controller 118, or inputted at a clinician device. In some aspects, the infusion order 201 comprises a bolus infusion order.

Workflow 200 then proceeds to block 204 with determining the infusion order 201 exceeds a bolus threshold. A bolus threshold comprises an operational threshold associated with the infusion device, e.g., infusion pump 116. For example, a bolus threshold may comprise a mechanical limit of the infusion device, such as a maximum or minimum flow rate, operating time, or the like. In some cases, the bolus threshold may be based on a state of the infusion device, and/or a coupled infusion device. For example, a bolus threshold may comprise a maximum volume for infusion based on a volume of fluid in a fluid container coupled to an infusion pump. As another example, a bolus threshold may be further based on one or more current infusions, such as a current infusion order for an infusion system. In some aspects, a set of bolus thresholds may be compared to the infusion order 201, for example, one or more of a flow rate threshold, a type of fluid threshold, a volume to be infused (VTBI), a total volume, or the like.

In some aspects, the infusion order 201 may be determined to exceed the bolus threshold by comparing a flow rate of the infusion order to the bolus threshold, where the bolus threshold is a flow rate threshold associated with a first infusion device coupled to the patient, and determining the flow rate of the infusion order exceeds the flow rate threshold. For example, a first infusion pump may have a flow rate threshold of 200 mL/hr, and the infusion order may have a flow rate of 400 mL/hr, thus, the infusion order flow rate is determined to exceed the flow rate threshold of the infusion pump.

In some aspects, the infusion order 201 may be determined to exceed the bolus threshold by comparing a total volume of the infusion order to the bolus threshold, where the bolus threshold is a total volume threshold associated with the first infusion device coupled to the patient, and determining the total volume of the infusion order exceeds the total volume threshold. For example, a first fluid container may have a total volume of 200 mL of fluid, and the infusion order may have a total volume of 300 mL of fluid, thus, the infusion order total volume is determined to exceed the total volume threshold of the fluid container.

Workflow 200 proceeds to block 206 with identifying a set of infusion devices associated with the patient, e.g., patient 110. In some aspects, the set of infusion devices may include devices of the infusion system 114 such as infusion pump 116 and/or fluid container 112. In some aspects, the set of infusion devices may include devices of two or more infusion systems, for example, infusion system 114 and a second infusion system coupled to the patient 110.

In some aspects, the set of infusion devices associated with the patient may be identified based on receiving an identifier of the first infusion device indicating the first infusion device is an infusion pump coupled to the patient, e.g., infusion pump 116 coupled to patient 110, and the identifier indicates one or more characteristics of the infusion pump. Characteristics of the infusion pump may include a bolus threshold, such as a flow rate threshold or a total volume threshold, a type of infusion pump, a current infusion order being infused by the infusion pump, a current status of the infusion pump, or the like.

Further, a second identifier may be received, indicating a second infusion device is coupled to the first infusion device, for example, a fluid container coupled to the infusion pump. The second identifier may indicate one or more characteristics of the fluid container, such as a total volume of the fluid in the fluid container, a size (e.g., volume) of the fluid container, a type of fluid contained in the fluid container, one or more other infusion devices coupled to the fluid container (e.g., another infusion pump coupled to the fluid container), or the like.

In some aspects, the second identifier may indicate a second infusion pump coupled to the patient, for example, a second pump module of the infusion system, or a second pump of a second infusion system coupled to the patient. Characteristics of the infusion pump may include a bolus threshold, such as a flow rate threshold or a total volume threshold, a type of infusion pump, a current infusion order being infused by the infusion pump, a current status of the infusion pump, or the like.

Workflow 200 then proceeds to block 208 with generating an infusion plan for allocating the infusion order between the set of infusion devices identified at block 206. In some aspects, two or more infusion plans may be generated at block 208. Generating infusion plan(s) is described in further detail with respect to workflow 300 in FIG. 3.

Optionally, workflow 200 proceeds to block 210 with displaying the infusion plan. For example, in some aspects, the infusion plan may be caused to be displayed on a graphical user interface associated with infusion pump controller 118, or on a graphical user interface associated with a clinician device. In some aspects, where multiple infusion plans are generated at block 208, one or more infusion plans may be displayed. For example, a list of multiple infusion plans may be displayed, or an indication of alternative infusion plans may be displayed.

Workflow 200 then proceeds to block 212 with receiving confirmation of the infusion plan. In some aspects, where multiple infusion plans are displayed, the confirmation may comprise a selection of one infusion plan of the list of multiple infusion plans.

Optionally, workflow 200 then proceeds to block 214 following block 212 or directly following block 208, with generating a clinical note comprising an indication of the infusion plan. In aspects where clinician confirmation is received, e.g., at block 212, the clinical note may include an indication of the clinician confirmation and/or a clinician identifier identifying the clinician who confirmed the infusion plan.

Workflow 200 then proceeds to block 216 with transmitting the clinical note to an EMR associated with the patient, for example using network 102 to transmit the clinical note to EMR system 122 in FIG. 1.

Workflow 200 then proceeds to block 218 with adjusting one or more operational parameters of the set of infusion devices according to the infusion plan. In some aspects, adjusting the one or more operational parameters of the set of infusion devices comprises adjusting an operational parameter of the first infusion device and adjusting an operational parameter of the second infusion device. For example, a flow rate of the first infusion device may be adjusted according to the infusion plan, and a flow rate of the second infusion device may be adjusted according to the infusion plan, whereby the flow rates of both the first and second infusion devices together may sum to be equal to the flow rate of the infusion order 201. In another example, a volume to be infused by a first infusion device may be adjusted according to the infusion plan, and a volume to be infused by a second infusion device may be adjusted according to the infusion plan, whereby the volume to be infused by the first and second infusion devices together may sum to be equal to the volume to be infused of the infusion order 201.

Note that workflow 200 is just one example, and other flows including fewer, additional, or alternative steps, consistent with this disclosure, are possible.

### Example Infusion Plan Generation Workflow

FIG. 3 depicts an example workflow 300 for generating an infusion plan, such as at block 208 of FIG. 2.

Initially, at block 302, an operational threshold associated with a first infusion device, e.g., identified at block 206 of workflow 200, may be compared to the infusion order 201. For example, where the operational threshold of the first infusion device is equal to or greater than the infusion order 201, the first operational threshold may be satisfied and workflow 300 proceeds to block 304.

In some aspects, an operational threshold may be associated with one operational parameter, and a set of operational thresholds (e.g., each associated with one operational parameter), may be compared to the infusion order. For example, a flow rate threshold may be compared to a flow rate of the infusion order, a volume to be infused may be compared to a volume to be infused of the infusion order, or the like.

At block 304, the first infusion device is added to the infusion plan. For example, an identifier of the first infusion device may be added to the infusion plan. An identifier may indicate which infusion device, for example, an infusion pump 116 of the infusion system 114, are to infuse the infusion order 201.

At block 306, the operational parameters of the first infusion device are determined based on the infusion order 201. For example, where the infusion order comprises 20 mL to be infused for 30 minutes, the flow rate and volume to be infused by the first infusion device may be determined as a flow rate of 40 mL/hr for a volume of 20 mL.

Workflow 300 then proceeds to block 308 with generating the infusion plan comprising the identifier of the first infusion device and the operational parameter(s) of the first infusion device. In some aspects, such as where an infusion plan component 106 is separate from the infusion device, e.g., infusion system 114, the generated infusion plan may be transmitted to the infusion device to implement the operational parameter(s).

Returning to block 302, where the operational threshold of the first infusion device is less than the infusion order 201, the first operational threshold may not be satisfied and workflow 300 proceeds to block 310. For example, a flow rate, volume to be infused, total volume, and/or the like of the infusion order 201 exceeds an operational threshold of the first infusion device.

At block 310, a second infusion device is identified from the set of infusion devices identified at block 206 of workflow 200.

Workflow 300 then proceeds to block 312 with determining whether the first operational threshold and a second operational threshold associated with the second infusion device are satisfied. In some aspects, the first and second operational thresholds may be combined to form a combination threshold associated with both the first infusion device and the second infusion device. For example, a first operational threshold may be a flow rate threshold of 400 mL/hr, and a second operational threshold may be a flow rate threshold of 200 mL/hr, the combination threshold may be 600 mL/hr. As another example, a first operational threshold may be a volume to be infused threshold of 500 mL, and a second operational threshold may be a volume of 300 mL, the combination threshold may be 800 mL.

If the first and second operational thresholds are not satisfied at block 312, then workflow 300 returns to block 310 with identifying another infusion device (e.g., a third or subsequent infusion device), until together the operational thresholds are satisfied at block 312.

Once the operational thresholds are satisfied, at block 312, workflow 300 proceeds to block 314 with adding the first and second, or any subsequent infusion devices, to the infusion plan. For example, an identifier of the first infusion device and an identifier of the second infusion device may be added to the infusion plan. An identifier may indicate which infusion devices, for example, an infusion pump 116 and a second infusion pump of the infusion system 114, are to infuse the infusion order 201. In some aspects, a second infusion device may comprise part of a second infusion system, and an identifier of the second infusion system may also be added to the infusion plan.

At block 316, the operational parameters of the first infusion device, the second infusion device, and any subsequent infusion device(s) are determined based on the infusion order 201. For example, where the infusion order comprises 200 mL to be infused for 30 minutes, the flow rate and volume to be infused by the first infusion device may be determined as a flow rate of 200 mL/hr for a volume of 100 mL and the second infusion device may be determined as a flow rate of 200 mL/hr for a volume of 100 mL.

The operational parameters may be further determined based on one or more characteristics of the infusion device(s), for example, operational thresholds of the infusion device, volume of fluid in a fluid container coupled to the infusion device, or the like.

In some aspects, one or more sets of operational parameters may be determined for the first, second, and any subsequent infusion device(s), such as alternative sets of operational parameters. For example, an infusion order may comprise an infusion of fluid A for 1 hour at a flow rate of 1200 mL/hr. Three infusion devices may be identified with a flow rate threshold of 999 mL/hr and each infusion device is coupled to a fluid container of 1000 mL of fluid A. A first set of operational parameters may comprise each infusion device infusing 400 mL of fluid A at 400 mL/hr. A second set of operational parameters may comprise a first infusion device infusing 600 mL of fluid A at 600 mL/hr and the other two infusion devices infusing 300 mL of fluid A at 300 mL/hr. A third set of operational parameters may comprises a first infusion device infusing 800 mL of fluid A at 800 mL/hr and the other two infusion devices infusing 200 mL of fluid A at 200 mL/hr. Other sets of operational parameters are possible, for example, additional or fewer.

Workflow 300 then proceeds to block 308 with generating the infusion plan comprising the identifier of the first infusion device, the identifier of the second infusion device, identifier of any subsequent infusion device(s) and the operational parameter(s) of the first, second, and subsequent infusion devices (if any).

In some aspects, such as where one or more sets of operational parameters are determined at block 316, each set of operational parameters may be used to generate an infusion plan. For example, a first set of operational parameters may be used to generate a first infusion plan, while a second set of operational parameters may be used to generate a second infusion plan. In some aspects, a priority level may be associated with each infusion plan, for example, a first priority level with the first infusion plan and a second priority level with a second infusion plan. As described at block 210, the infusion plans may be displayed based on priority level, for example, a higher priority infusion plan displayed first or at the top of a list.

A priority level may be assigned based on one or more characteristics of the infusion device, such as flow rate threshold, volume of a coupled fluid container, expiration of a fluid, expiration of an infusion administration set associated with the infusion device, a location or configuration of the infusion device, or the like. For example, operational parameters closer to an operational threshold may be assigned a lower priority compared to operational parameters further from the operational threshold. As another example, an operational parameter which may deplete a volume of fluid in a container may be assigned a higher priority level compared to an operational parameter which will not deplete a volume of fluid in a fluid container. Fluid expiring or an infusion administration set expiring may be assigned a higher priority. An infusion device at a particular position in a configuration, e.g., an infusion pump closer to an infusion controller, may be assigned a higher or lower priority. In some aspects, priority level assignment may be configured based on a hospital information system, for example, for an entire medical center, a care area of the medical center, patient type, or the like.

Note that workflow 300 is just one example, and other flows including fewer, additional, or alternative steps, consistent with this disclosure, are possible.

### Example Infusion Plan

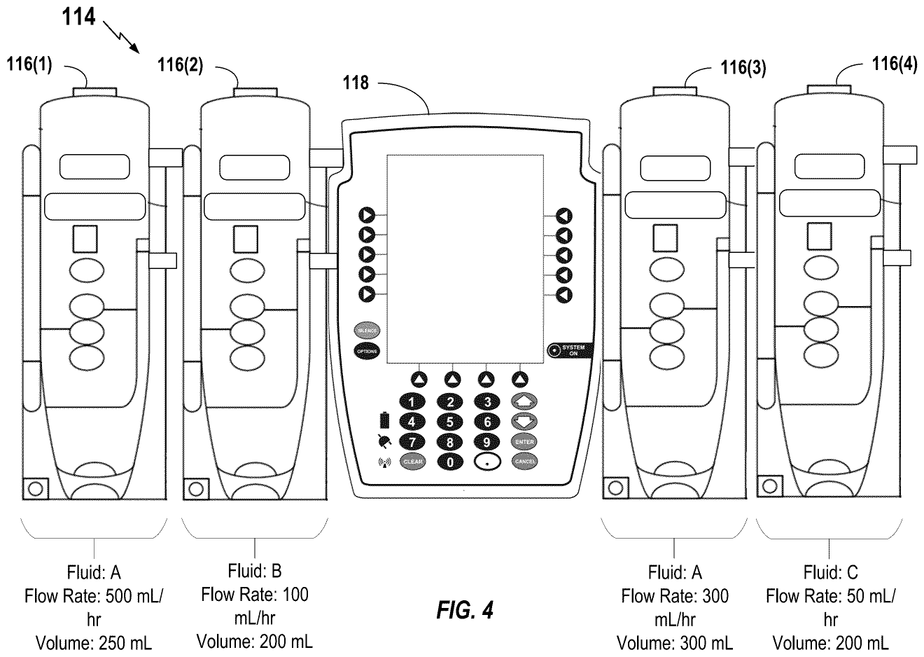

FIG. 4 depicts an example infusion plan for an infusion system 114. In this example, infusion system includes infusion pump controller 118, and four infusion pumps, infusion pump 116(1), infusion pump 116(2), infusion pump 116(3), and infusion pump 116(4), collectively infusion pumps 116.

Infusion pump 116(1) is coupled to a fluid container (not depicted) containing fluid A, and has a fluid volume threshold of 250 mL (e.g., volume of fluid A available for infusion). Infusion pump 116(1) also has a flow rate threshold of 500 mL/hr.

Infusion pump 116(2) is coupled to a fluid container (not depicted) containing fluid B, and has a fluid volume threshold of 200 mL (e.g., volume of fluid A available for infusion). Infusion pump 116(2) also has a flow rate threshold of 100 mL/hr.

Infusion pump 116(3) is coupled to a fluid container (not depicted) containing fluid A, and has a fluid volume threshold of 300 mL (e.g., volume of fluid A available for infusion). Infusion pump 116(3) also has a flow rate threshold of 500 mL/hr.

Infusion pump 116(4) is coupled to a fluid container (not depicted) containing fluid C, and has a fluid volume threshold of 50 mL (e.g., volume of fluid A available for infusion). Infusion pump 116(4) also has a flow rate threshold of 200 mL/hr.

For example, an infusion order may be for an infusion of fluid A, 400 mL over 30 minutes. The operational parameter for infusing 400 mL of fluid A over 30 minutes with a single infusion pump may be a flow rate of 800 mL/hr, a volume to be infused of 400 mL of fluid A, for a time of 30 minutes.

For an infusion order for fluid A, infusion pump 116(1) and infusion pump 116(3) may be identified as part of the set of infusion devices available for the infusion plan. Infusion pump 116(2) and infusion pump 116(4) are not coupled to fluid A, and thus may not be used.

Infusion pump 116(1) has a flow rate threshold of 500 mL/hr, which is less than the operational flow rate of 800 mL/hr for the infusion order, e.g., as determined at block 302 of workflow 300. Infusion pump 116(3) has a flow rate threshold of 300 mL/hr, which is less than the operational flow rate of 800 mL/hr for the infusion order, e.g., as determined at block 302 of workflow 300.

However, together, infusion pump 116(1) and infusion pump 116(3) working in tandem have a flow rate threshold of 800 mL/hr, which does satisfy the infusion order flow rate of 800 mL/hr, e.g., as determined at block 312 of workflow 300. Both infusion pump 116(1) and infusion pump 116(3) may be added to the infusion plan, e.g., at block 314 of workflow 300.

Infusion order may then be allocated between infusion pump 116(1) and infusion pump 116(3), e.g., at block 316 of workflow 300. For example, infusion pump 116(1) may be determined to infuse fluid A at a flow rate of 500 mL/hr for a volume to be infused of 250 mL for 30 minutes. Infusion pump 116(3) may be determined to infuse fluid A at a flow rate of 300 mL/hr for a volume to be infused of 150 mL for 30 minutes. Thus, together, infusion pump 116(1) and infusion pump 116(3) will infuse a total volume of 400 mL of fluid A over 300 minutes.

### Example Infusion System

FIG. 5 depicts an example infusion system 500, which may implement methods described herein for generating and implementing infusion plans. The infusion system 500 may be coupled to a patient 550 to administer a substance to the patient 150 through infusion. The infusion system 500 may include one or more pumps, for example, pump 502, pump 504, pump 506, and pump 508. Although a large volume pump is illustrated, other types of pumps may be implemented, such as a peristaltic pump, a small volume pump, a syringe pump, an anesthesia delivery pump, or a patient-controlled analgesic. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 502, 504, 506, or 508 may comprise a standard infusion pumping unit, patient-controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+ ml/hr).

Each of the pumps 502, 504, 506, or 508 may be fluidly connected with an upstream fluid line 512, fluid line 514, fluid line 516, and fluid line 518, respectively. Further, each of pump 502, pump 504, pump 506, and pump 508, may be fluidly connected with a downstream fluid line 522, fluid line 524, fluid line 526, and fluid line 528, respectively. The fluid lines may be any type of fluid conduit, such as tubing, through which fluid can flow.

Each of fluid supply 532, fluid supply 534, fluid supply 536, and fluid supply 538, may be a reservoir, for example, as bottles shown, inverted and suspended above the pumps. Fluid supplies may also take the form of bags, syringes, or other types of containers. The infusion system 500 may be mounted on a roller stand or intravenous pole 540.

Infusion system 500 may further comprise check valves, drip chambers, valved ports, connectors, and other devices configured to administer a substance.

Each of the pumps 502, 504, 506, or 508 of infusion system 500 may be coupled to an infusion pump controller 560. The infusion pump controller 560 may be a patient care device (PCD) or patient care unit (PCU). The infusion pump controller 560 is configured to control operations of the pumps 502, 504, 506, or 508, for example, to control infusions to patient 550. The infusion pump controller 560 may be further configured to the control of fluid delivery to the patient and the monitoring of the fluid path for occlusion or air-in-line obstructions.

The infusion pump controller 560 may further include a graphical user interface (GUI), for example, an information display such as a liquid crystal display, and/or a touch-screen. The user interface of the infusion pump controller 560 may be used during set up and/or operating procedures to facilitate control of the infusion system 500. For example, entry, editing, and/or display of operational parameters of the pumps 502, 504, 506, or 508. The infusion pump controller 560 may further include one or more softkeys and/or hardkeys to facilitate data entry and commands. In some aspects, a user interface may display the operational parameters of the pumps 502, 504, 506, or 508, for example, the infusion rate at which the pump is operating. The user interface may additionally and/or alternatively, be used to display informational, advisory, alarm, or malfunction messages associated with the pumps 502, 504, 506, or 508.

The infusion pump controller 560 may further be configured to provide power to the infusion system 500 and interface between aspects of the infusion system 500 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the infusion pump controller 560 is configured to provide physical attachment of the infusion system 500 to structures, such as intravenous pole 540 and/or bedrails, or the like.

### Example Syringe Pump

FIG. 6 depicts an example syringe pump 602. Syringe pump 602 includes a graphical user interface (GUI) 624 for displaying operational information, including the selected syringe type and the configured infusion (e.g., in terms of rate of infusion, volume infused, length of time of infusion, etc.). A user may operate syringe pump 602 through one or more buttons 622 in this example. Note that other examples may include other arrangements, such as touch-screen interfaces, or interfaces operable by remote device, such as by an application on a computing device.

Syringe 601 is shown next to syringe pump 602, rather than loaded in the pump, for clarity of illustration. Syringe pump 602 includes cradle 604 in which a barrel 603 of syringe 601 rests when mounted in the syringe pump 602. Sensor 628 detects volume markings along a barrel of syringe 601. Cradle 604 has a clamp 606 to securely hold the barrel 603 in a fixed position in cradle 604 to resist axial and lateral movement. Clamp 606 may pivot between an open position to permit loading or removal of syringe 601 and a closed position over cradle 604. Barrel clamp 606 may measure an outside diameter of the barrel 603 of syringe 601. Barrel flange 605 of syringe 601 resides in a barrel flange groove 608 in syringe pump 602 to immobilize barrel 603 from axial movement during movement of plunger 607 within barrel 603 of syringe 601. Barrel flange groove may measure an outside diameter and a thickness of the barrel flange 605 of syringe 601.

Plunger 607 can include push-button 609 having an inner side 611 and being interconnected with stopper 613 of plunger 607 by piston 615. When mounted in syringe pump 602, push-button 609 can be held by drive head 610 with a plunger retainer comprising a pair of pivotally mounted claws, first retainer claw 612 and second retainer claw 614, shown in the closed position in FIG. 6. The retainer claws 612 and 614 can curve inwardly toward each other to grasp push-button 609 mounted in syringe pump 602.

A rotation knob 616 can be used to control the positions of the first and second retainer claws 612 and 614 to allow removal and insertion of the push-button 609 and to release the split-nut from the driveshaft to permit axial positioning of the drive head 610. Syringes can be provided for use with a syringe pump with different quantities of fluid, and the plunger can be located at different positions in relation to the barrel.

The drive head 610 can allow manual adjustment to accommodate syringes with different beginning plunger positions. A syringe inserted in the cradle 604 can align with the drive head 610 within a particular axial range. The points where the axial center lines of the syringe intersect the driver can change according to the size of the syringe but only in one direction along the drive head 610. A guide device 618 can extend from the drive head 610 to a point within a body of the syringe pump 602.

Syringe pump 602 can include a control panel 620 providing multiple buttons 622 for control of the syringe pump 602 as well as GUI 624 used to present pump-specific information to the operator. The buttons 622 can allow the operator to program the syringe pump 602 for the flow rate, the volume to be infused, and other pump parameters. GUI 624 can present the programmed flow rate, the amount of fluid remaining to be infused, as well as alarms and other information.

The drive head 610 can include a contact plate 626 that has a pushing surface that contacts the outer side 617 of the push-button 609 as the drive head 610 moves forward toward the barrel 603, pushing the plunger 607 into the barrel 603 of the syringe to expel the syringe contents through a fluid administration set tubing 619 to a patient. A drive mechanism 654 is configured to drive the drive head 610 forward. The drive mechanism 654 may include, a drivetrain, for example, gears, axles, shafts, chains, hydraulics, and/or any other components for translating rotational motion of a motor into linear motion of the drive head 610. The drivetrain may include linear actuating components, such as a linear stepper motor, a brushed DC electric motor, a brushless DC electric motor, a servo motor, an AC motor, or any other type of motor.

When the contact plate 626 exerts force against the push-button 609, the force may be detected by force sensor 629 and transmitted to a processor for monitoring. In the case of friction between the stopper 613 and the barrel 603, the force exerted can increase and can be detected by force sensor 629.

Syringe pump 602 is configured with known syringe types containing information such as syringe inner diameter and stroke of the stopper 613. Syringe pump 602 determines the position of stopper 613 based on the movement of drive head 610 based on the syringe type and stored characteristics of the syringe type. Then, syringe pump 602 calculates the volume infused, time elapsed, volume remaining, and time remaining. As the drive head 610 continues to move, a flow rate is determined based on the characteristics of the syringe and the velocity of the drive head 610.

One or more sensors, such as sensor 628, barrel clamp 606, groove 608, claws 612 and 614, and force sensor 629, may measure various characteristics of syringe 601, to obtain a measured value for the characteristic, such as barrel length, barrel outside diameter, flange shape, flange size, plunger shape, plunger size (e.g., diameter), plunger tab thickness, volume per plunger distance moved, driver head height, etc. These sensors may include one or more of an optical sensor, a light source, a pressure sensor, a position sensor (e.g., magnetic linear position sensor indicating the plunger head), force sensor, or the like.

### Example Method for Infusion Order Allocation

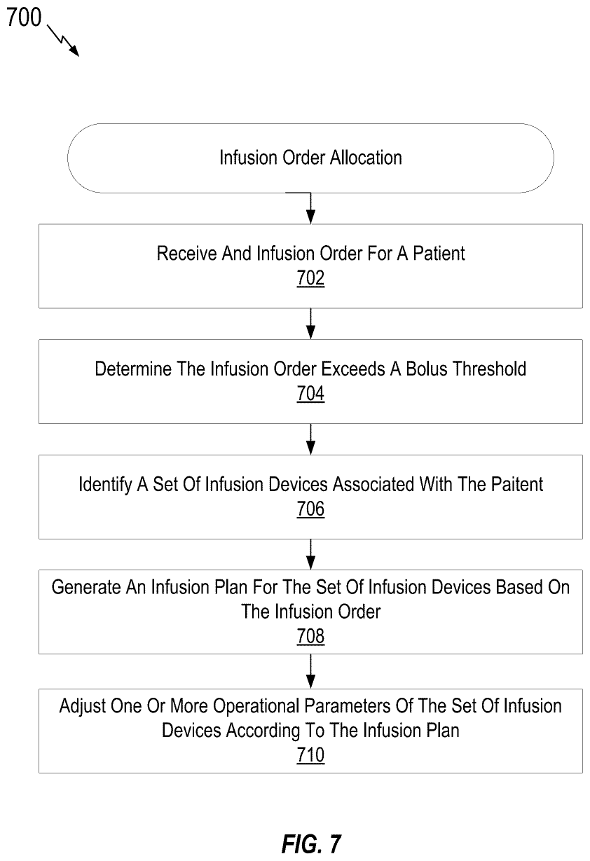

FIG. 7 shows an example method 700 for infusion order allocation.

Initially, method 700 begins at step 702 with receiving an infusion order for a patient, such as described at block 202 in FIG. 2.

Method 700 proceeds at step 704 with determining the infusion order exceeds a bolus threshold, such as described at block 204 in FIG. 2.

In some aspects, determining the infusion order exceeds the bolus threshold, comprises: comparing a flow rate of the infusion order to the bolus threshold, wherein the bolus threshold comprises a flow rate threshold associated with a first infusion device of the set of infusion devices; and determining the flow rate of the infusion order exceeds the flow rate threshold associated with the first infusion device of the set of infusion devices.

In some aspects, determining the infusion order exceeds the bolus threshold, comprises: comparing a volume of the infusion order to the bolus threshold, wherein the bolus threshold comprises a volume threshold associated with a first infusion device of the set of infusion devices; and determining the volume of the infusion order exceeds the volume threshold associated with the first infusion device of the set of infusion devices.

Method 700 then proceeds at step 706 with identifying a set of infusion devices associated with the patient, such as described at block 206 in FIG. 2.

In some aspects, identifying the set of infusion devices associated with the patient, comprises: receiving, an identifier of a first infusion device of the set of infusion devices, wherein: the identifier indicates the first infusion device is an infusion pump coupled to the patient, and the identifier indicates one or more characteristics of the infusion pump; and receiving, an identifier of a second infusion device of the set of infusion devices, wherein: the identifier indicates the second infusion device is a fluid container coupled to the infusion pump, the identifier indicates one or more characteristics of the fluid container.

In some aspects, the set of infusion devices associated with the patient comprises at least one infusion pump coupled to the patient. In some aspects, the set of infusion devices associated with the patient comprises at least one infusion system coupled to the patient. In some aspects, the first infusion device comprises a first infusion pump, and the second infusion device comprises a second infusion pump. In some aspects, the first infusion device comprises a first infusion system, and the second infusion device comprises a second infusion system.

Method 700 proceeds at step 708 with generating an infusion plan for the set of infusion devices based on the infusion order, such as described at block 208 in FIG. 2.

In some aspects, generating the infusion plan for the set of infusion devices based on the infusion order, comprises: determining the infusion order satisfies a first operational threshold associated with a first infusion device of the set of infusion devices, such as described at block 302 in FIG. 3; and adding the first infusion device to the infusion plan, wherein adjusting the one or more operational parameters comprises adjusting at least one operational parameter of the first infusion device, such as described at block 304 in FIG. 3.

In some aspects, generating the infusion plan for the set of infusion devices based on the infusion order, comprises: determining the infusion order exceeds a first operational threshold associated with a first infusion device of the set of infusion devices, such as described at block 302 in FIG. 3; identifying a second infusion device of the set of infusion devices, such as described at block 310 in FIG. 3; determining the infusion order satisfies a second operational threshold associated with a combination of the first infusion device and the second infusion device, such as described at block 312 in FIG. 3; and adding the first infusion device and the second infusion device to the infusion plan, wherein adjusting the one or more operational parameters comprises adjusting at least one operational parameter of the first infusion device and at least one operational parameter of the second infusion device, such as described at block 314 in FIG. 3.

In some aspects, the infusion plan further comprises information indicating the one or more operational parameters of the set of infusion devices, and the method further comprises: in response to adding the first infusion device and the second infusion device to the infusion plan, determining the at least one operational parameter of the first infusion device and the at least one operational parameter of the second infusion device, such as described at block 316 in FIG. 3.

In some aspects, the at least one operational parameter of the first infusion device is determined based on a volume of fluid in a first fluid container associated with the first infusion device and the at least one operational parameter of the first infusion device comprises a volume of fluid to be infused by the first infusion device, the at least one operational parameter of the second infusion device is based on a volume of fluid in a second fluid container associated with the second infusion device and the at least one operational parameter of the second infusion device comprises a volume of fluid to be infused by the second infusion device, and a sum of the volume of fluid to be infused by the first infusion device and the volume of fluid to be infused by the second infusion device equals a total volume of fluid of the infusion order.

In some aspects, the at least one operational parameter of the first infusion device is determined based on a flow rate of the first infusion device and the at least one operational parameter of the first infusion device comprises a flow rate of fluid to be infused by the first infusion device, the at least one operational parameter of the second infusion device is based on a flow rate of the second infusion device and the at least one operational parameter of the second infusion device comprises a flow rate of fluid to be infused by the second infusion device, and a sum of the flow rate of fluid to be infused by the first infusion device and the flow rate of fluid to be infused by the second infusion device equals a flow rate of fluid of the infusion order.

In some aspects, the first operational threshold is associated with a maximum volume of fluid to be infused by the first infusion device. In some aspects, the first operational threshold is associated with a flow rate of fluid to be infused by the first infusion device.

In some aspects, the second operational threshold is associated with a maximum volume of fluid to be infused by the combination of the first infusion device and the second infusion device. In some aspects, the second operational threshold is associated with a flow rate of fluid to be infused by the combination of the first infusion device and the second infusion device. In some aspects, the second infusion device is associated with a fluid container containing a fluid of the infusion order.

Method 700 then proceeds at step 710 with adjusting one or more operational parameters of the set of infusion devices according to the infusion plan, such as described at block 218 in FIG. 2.

In some aspects, method 700 further comprises causing, on a user interface associated with the set of infusion devices, display of the infusion plan; and receiving, in response to the infusion plan, a confirmation to adjust the set of infusion devices, wherein the one or more operational parameters are adjusted in response to receiving the confirmation.

In some aspects, method 700 further comprises generating, based on the infusion plan and the one or more operational parameters, a clinical note; and transmitting, to an electronic medical record system, the clinical note.

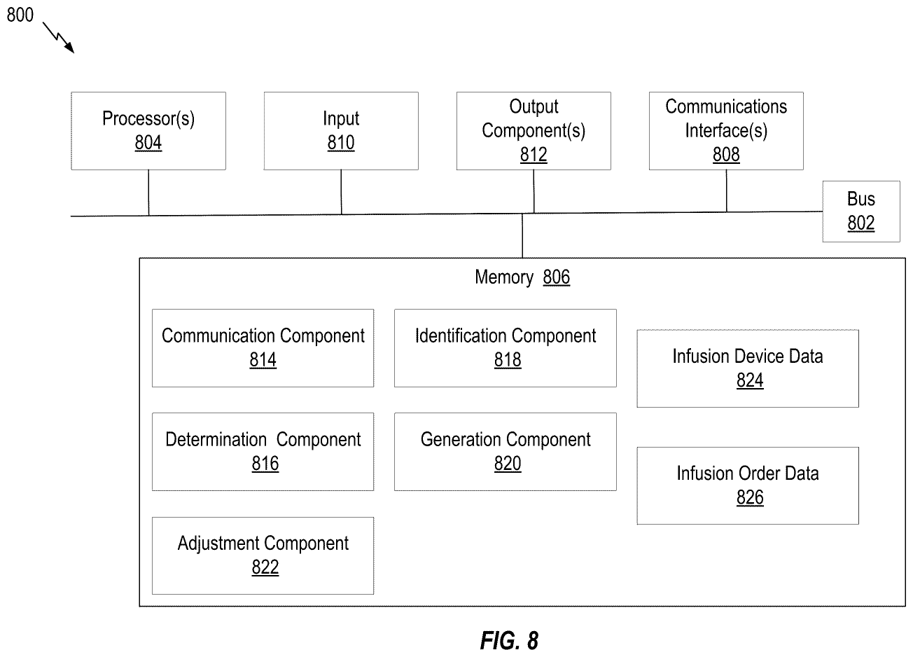

In one aspect, method 700, or any aspect related to it, may be performed by an apparatus, such as computing device 800 of FIG. 8, which includes various components operable, configured, or adapted to perform the method 700. Computing device 800 is described below in further detail.

Note that FIG. 7 is just one example of a method, and other methods including fewer, additional, or alternative steps are possible consistent with this disclosure.

### Example Computing Device for Allocating Infusion Orders

FIG. 8 depicts an example computing device 800, such as a medical device, which implements various features and processes described herein, such as infusion system 500 in FIG. 5 and/or syringe pump 602. For example, the computing device 800 may perform one or more steps of any of workflow 200, workflow 300 or method 700. The computing device 800 may include one or more processors 804, one or more memories 806, one or more input components 810, one or more output components 812, and one or more communication interfaces 808. Each of these components may be coupled by a bus 802.

Computing device 800 may perform these processes based on one or more processors 804 executing software instructions stored by a computer-readable medium, such as one or more memories 806. In certain embodiments, one or more processors 804 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 808. When executed, software instructions stored in one or more memories may cause one or more processors 804 to perform one or more processes described herein.

A memory 806 may include data storage or one or more data structures (e.g., a database, etc.). Computing device 800 may be capable of receiving information from, storing information in, communicating information to, or searching information stored in the data storage or one or more data structures in one or more memories 806.

A memory 806 may include random access memory (RAM), read only memory (ROM), and/or other types of dynamic or static storage devices (e.g., flash memory, magnetic memory, optical memory, etc.), that stores information and/or instructions for use by one or more processors 804. For example, a memory 806 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 806 may include a communication component 814 is configured for receiving an infusion order for a patient, such as described at block 202 in FIG. 2, and step 702 of FIG. 7, including infusion order data 826.

In some aspects, one or more memories 806 may include a determination component 816 is configured for determining the infusion order exceeds a bolus threshold, such as described at block 204 in FIG. 2, and step 704 of FIG. 7.

In some aspects, one or more memories 806 may include an identification component 818 is configured for identifying a set of infusion devices associated with the patient, such as described at block 206 in FIG. 2, and step 706 of FIG. 7, including using infusion device data 824.

In some aspects, one or more memories 806 may include a generation component 820 configured for generating an infusion plan for the set of infusion devices based on the infusion order, such as described at block 208 in FIG. 2 and step 708 of FIG. 7.

In some aspects, one or more memories 806 may include an adjustment component 822 configured for adjusting one or more operational parameters of the set of infusion devices according to the infusion plan, such as described at block 218 in FIG. 2, and step 710 of FIG. 6

One or more processors 804 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 810 may include a component that permits computing device 600 to receive information, such as via user input (e.g., a touch-screen display, a keyboard, a keypad, a mouse, a button, a switch, a microphone, etc.). Further, one or more input components 810 may include a sensor for sensing information (e.g., a global positioning system (GPS) component, an accelerometer, a gyroscope, an actuator, etc.).

One or more output components 812 may include a component that provides output information from computing device 800 (e.g., a display, a speaker, one or more light-emitting diodes (LEDs), etc.).

Communication interface 808 may include a transceiver-like component (e.g., a transceiver, a separate receiver and transmitter, etc.) that enables computing device 800 to communicate with other devices, such as via a wired connection, a wireless connection, or a combination of wired and wireless connections. Communication interface 808 may permit computing device 800 to receive information from another device and/or provide information to another device. For example, communication interface 808 may include an Ethernet interface, an optical interface, a coaxial interface, an infrared interface, a radio frequency (RF) interface, a universal serial bus (USB) interface, a Wi-Fi interface, a cellular network interface, and/or the like.

### Example Clauses

Implementation examples are described in the following numbered clauses:
Clause 1: A method, comprising: receiving an infusion order for a patient; determining the infusion order exceeds a bolus threshold; identifying a set of infusion devices associated with the patient; generating an infusion plan for the set of infusion devices based on the infusion order; and adjusting one or more operational parameters of the set of infusion devices according to the infusion plan.
Clause 2: The method of clause 1, wherein determining the infusion order exceeds the bolus threshold, comprises: comparing a flow rate of the infusion order to the bolus threshold, wherein the bolus threshold comprises a flow rate threshold associated with a first infusion device of the set of infusion devices; and determining the flow rate of the infusion order exceeds the flow rate threshold associated with the first infusion device of the set of infusion devices.
Clause 3: The method of any one of clauses 1-2, wherein determining the infusion order exceeds the bolus threshold, comprises: comparing a volume of the infusion order to the bolus threshold, wherein the bolus threshold comprises a volume threshold associated with a first infusion device of the set of infusion devices; and determining the volume of the infusion order exceeds the volume threshold associated with the first infusion device of the set of infusion devices.
Clause 4: The method of any one of clauses 1-3, wherein identifying the set of infusion devices associated with the patient, comprises: receiving, an identifier of a first infusion device of the set of infusion devices, wherein: the identifier indicates the first infusion device is an infusion pump coupled to the patient, and the identifier indicates one or more characteristics of the infusion pump; and receiving, an identifier of a second infusion device of the set of infusion devices, wherein: the identifier indicates the second infusion device is a fluid container coupled to the infusion pump, the identifier indicates one or more characteristics of the fluid container.
Clause 5: The method of any one of clauses 1-4, wherein generating the infusion plan for the set of infusion devices based on the infusion order, comprises: determining the infusion order satisfies a first operational threshold associated with a first infusion device of the set of infusion devices; and adding the first infusion device to the infusion plan, wherein adjusting the one or more operational parameters comprises adjusting at least one operational parameter of the first infusion device.
Clause 6: The method of any one of clauses 1-5, wherein generating the infusion plan for the set of infusion devices based on the infusion order, comprises: determining the infusion order exceeds a first operational threshold associated with a first infusion device of the set of infusion devices; identifying a second infusion device of the set of infusion devices; determining the infusion order satisfies a second operational threshold associated with a combination of the first infusion device and the second infusion device; and adding the first infusion device and the second infusion device to the infusion plan, wherein adjusting the one or more operational parameters comprises adjusting at least one operational parameter of the first infusion device and at least one operational parameter of the second infusion device.
Clause 7: The method of clause 6, wherein the infusion plan further comprises information indicating the one or more operational parameters of the set of infusion devices, and the method further comprises: in response to adding the first infusion device and the second infusion device to the infusion plan, determining the at least one operational parameter of the first infusion device and the at least one operational parameter of the second infusion device.
Clause 8: The method of clause 7, wherein: the at least one operational parameter of the first infusion device is determined based on a volume of fluid in a first fluid container associated with the first infusion device and the at least one operational parameter of the first infusion device comprises a volume of fluid to be infused by the first infusion device, the at least one operational parameter of the second infusion device is based on a volume of fluid in a second fluid container associated with the second infusion device and the at least one operational parameter of the second infusion device comprises a volume of fluid to be infused by the second infusion device, and a sum of the volume of fluid to be infused by the first infusion device and the volume of fluid to be infused by the second infusion device equals a total volume of fluid of the infusion order.
Clause 9: The method of clause 7, wherein: the at least one operational parameter of the first infusion device is determined based on a flow rate of the first infusion device and the at least one operational parameter of the first infusion device comprises a flow rate of fluid to be infused by the first infusion device, the at least one operational parameter of the second infusion device is based on a flow rate of the second infusion device and the at least one operational parameter of the second infusion device comprises a flow rate of fluid to be infused by the second infusion device, and a sum of the flow rate of fluid to be infused by the first infusion device and the flow rate of fluid to be infused by the second infusion device equals a flow rate of fluid of the infusion order.
Clause 10: The method of any of clauses 5-7, wherein the first operational threshold is associated with a maximum volume of fluid to be infused by the first infusion device.
Clause 11: The method of any of clauses 5-7, wherein the first operational threshold is associated with a flow rate of fluid to be infused by the first infusion device.
Clause 12: The method of clause 6, wherein the second operational threshold is associated with a maximum volume of fluid to be infused by the combination of the first infusion device and the second infusion device.
Clause 13: The method of clause 6, wherein the second operational threshold is associated with a flow rate of fluid to be infused by the combination of the first infusion device and the second infusion device.
Clause 14: The method of clause 6, wherein the second infusion device is associated with a fluid container containing a fluid of the infusion order.
Clause 15: The method of any one of clauses 1-14, wherein the set of infusion devices associated with the patient comprises at least one infusion pump coupled to the patient.
Clause 16: The method of any one of clauses 1-15, wherein the set of infusion devices associated with the patient comprises at least one infusion system coupled to the patient.
Clause 17: The method of clause 6, wherein: the first infusion device comprises a first infusion pump, and the second infusion device comprises a second infusion pump.
Clause 18: The method of clause 6, wherein: the first infusion device comprises a first infusion system, and the second infusion device comprises a second infusion system.
Clause 19: The method of any one of clauses 1-18, further comprising: causing, on a user interface associated with the set of infusion devices, display of the infusion plan; and receiving, in response to the infusion plan, a confirmation to adjust the set of infusion devices, wherein the one or more operational parameters are adjusted in response to receiving the confirmation.
Clause 20: The method of any one of clauses 1-19, further comprising: generating, based on the infusion plan and the one or more operational parameters, a clinical note; and transmitting, to an electronic medical record system, the clinical note.
Clause 21: An infusion system, comprising: an infusion pump configured to deliver fluid to a patient; and an infusion pump controller configured to perform a method in accordance with any one of Clauses 1-20.
Clause 22: A processing system, comprising: memory comprising computer-executable instructions; and one or more processors configured to execute the computer-executable instructions and cause the processing system to perform a method in accordance with any one of Clauses 1-20.
Clause 23: A processing system, comprising means for performing a method in accordance with any one of Clauses 1-20.
Clause 24: A non-transitory computer-readable medium storing program code for causing a processing system to perform the steps of any one of Clauses 1-20.
Clause 25: A computer program product embodied on a computer-readable storage medium comprising code for performing a method in accordance with any one of Clauses 1-20.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database or another data structure), ascertaining, and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An infusion system, comprising:
an infusion pump configured to deliver fluid to a patient; and
an infusion pump controller configured to:
receive an infusion order for the patient;
determine the infusion order exceeds a bolus threshold;
identify a set of infusion devices associated with the patient, wherein the set of infusion devices comprises the infusion system;
generate an infusion plan for the set of infusion devices based on the infusion order; and
adjust one or more operational parameters of the set of infusion devices according to the infusion plan.

2. The infusion system of claim 1, wherein to determine the infusion order exceeds the bolus threshold, the infusion pump controller is further configured to:
compare a flow rate of the infusion order to the bolus threshold, wherein the bolus threshold comprises a flow rate threshold associated with a first infusion device of the set of infusion devices; and
determine the flow rate of the infusion order exceeds the flow rate threshold associated with the first infusion device of the set of infusion devices.

3. The infusion system of claim 1, wherein to determine the infusion order exceeds the bolus threshold, the infusion pump controller is further configured to:
compare a volume of the infusion order to the bolus threshold, wherein the bolus threshold comprises a volume threshold associated with a first infusion device of the set of infusion devices; and
determine the volume of the infusion order exceeds the volume threshold associated with the first infusion device of the set of infusion devices.

4. The infusion system of claim 1, wherein to identify the set of infusion devices associated with the patient, the infusion pump controller is further configured to:
receive, an identifier of a first infusion device of the set of infusion devices, wherein:
the identifier indicates the first infusion device is the infusion pump coupled to the patient, and
the identifier indicates one or more characteristics of the infusion pump; and
receive, an identifier of a second infusion device of the set of infusion devices, wherein:
the identifier indicates the second infusion device is a fluid container coupled to the infusion pump,
the identifier indicates one or more characteristics of the fluid container.

5. The infusion system of claim 1, wherein to generate the infusion plan for the set of infusion devices based on the infusion order, the infusion pump controller is further configured to:
determine the infusion order satisfies a first operational threshold associated with a first infusion device of the set of infusion devices, wherein the first infusion device is the infusion pump; and
add the first infusion device to the infusion plan, wherein adjusting the one or more operational parameters comprises adjusting at least one operational parameter of the first infusion device.

6. The infusion system of claim 1, wherein to generate the infusion plan for the set of infusion devices based on the infusion order, the infusion pump controller is further configured to:
determine the infusion order exceeds a first operational threshold associated with a first infusion device of the set of infusion devices;
identify a second infusion device of the set of infusion devices;
determine the infusion order satisfies a second operational threshold associated with a combination of the first infusion device and the second infusion device; and
add the first infusion device and the second infusion device to the infusion plan, wherein adjust the one or more operational parameters comprises adjusting at least one operational parameter of the first infusion device and at least one operational parameter of the second infusion device.

7. The infusion system of claim 6, wherein:
the infusion plan further comprises information indicating the one or more operational parameters of the set of infusion devices, and
in response to addition of the first infusion device, and the second infusion device to the infusion plan, the infusion pump controller is further configured to:
determine the at least one operational parameter of the first infusion device and the at least one operational parameter of the second infusion device.

8. The infusion system of claim 7, wherein:
the at least one operational parameter of the first infusion device is determined based on a volume of fluid in a first fluid container associated with the first infusion device and the at least one operational parameter of the first infusion device comprises a volume of fluid to be infused by the first infusion device,
the at least one operational parameter of the second infusion device is based on a volume of fluid in a second fluid container associated with the second infusion device and the at least one operational parameter of the second infusion device comprises a volume of fluid to be infused by the second infusion device, and
a sum of the volume of fluid to be infused by the first infusion device and the volume of fluid to be infused by the second infusion device equals a total volume of fluid of the infusion order.

9. The infusion system of claim 7, wherein:
the at least one operational parameter of the first infusion device is determined based on a flow rate of the first infusion device and the at least one operational parameter of the first infusion device comprises a flow rate of fluid to be infused by the first infusion device,
the at least one operational parameter of the second infusion device is based on a flow rate of the second infusion device and the at least one operational parameter of the second infusion device comprises a flow rate of fluid to be infused by the second infusion device, and
a sum of the flow rate of fluid to be infused by the first infusion device and the flow rate of fluid to be infused by the second infusion device equals a flow rate of fluid of the infusion order.

10. The infusion system of claim 5, wherein the first operational threshold is associated with a maximum volume of fluid to be infused by the first infusion device.

11. The infusion system of claim 5, wherein the first operational threshold is associated with a flow rate of fluid to be infused by the first infusion device.

12. The infusion system of claim 6, wherein the second operational threshold is associated with a maximum volume of fluid to be infused by the combination of the first infusion device and the second infusion device.

13. The infusion system of claim 6, wherein the second operational threshold is associated with a flow rate of fluid to be infused by the combination of the first infusion device and the second infusion device.

14. The infusion system of claim 6, wherein the second infusion device is associated with a fluid container containing a fluid of the infusion order.

15. The infusion system of claim 1, wherein the set of infusion devices associated with the patient comprises at least one infusion pump coupled to the patient.

16. The infusion system of claim 1, wherein the set of infusion devices associated with the patient comprises at least one infusion system coupled to the patient.

17. The infusion system of claim 6, wherein:
the first infusion device comprises a first infusion pump, and the second infusion device comprises a second infusion pump.

18. The infusion system of claim 6, wherein:
the first infusion device comprises a first infusion system, and the second infusion device comprises a second infusion system.

19. The infusion system of claim 1, wherein the infusion pump controller is further configured to:
causing, on a user interface associated with the set of infusion devices, display of the infusion plan; and
receiving, in response to the infusion plan, a confirmation to adjust the set of infusion devices, wherein the one or more operational parameters are adjusted in response to receiving the confirmation.

20. The infusion system of claim 1, wherein the infusion pump controller is further configured to:
generate, based on the infusion plan and the one or more operational parameters, a clinical note; and
transmit, to an electronic medical record system, the clinical note.
